(19) United States
(12) Patent Application Publication
(10) Pub. No.: US 2016/0243107 A1
(43) Pub. Date: Aug. 25, 2016

(54) Opioid Antagonist Formulations

(71) Applicant: PURDUE PHARMA L.P., Stamford, CT (US)

(72) Inventor: Margaret Moline, White Plains, NY (US)

(21) Appl. No.: 15/030,709

(22) PCT Filed: Dec. 22, 2014
(86) PCT No.: PCT/US14/71802

§ 371 Date: Apr. 20, 2016

Related U.S. Application Data
(60) Provisional application No. 61/920,067, filed on Dec. 23, 2013.

(51) Int. Cl. A61K 31/485; A61K 9/24; A61K 9/20

(52) U.S. Cl. CPC A61K 31/485; A61K 9/2031; A61K 9/209

(57) ABSTRACT

In certain embodiments, the present invention is directed to a solid controlled-release dosage form comprising a core comprising a core portion of an opioid antagonist and a shell encasing the core and comprising a shell portion of the opioid antagonist, wherein the release profile of the core portion of opioid antagonist is different than the release profile of the shell portion of opioid antagonist.

### FIELD OF THE INVENTION

The present invention relates to a pharmaceutical formulation comprising an opioid antagonist. In certain embodiments, the formulation provides abuse deterrence and/or treatment of an opioid agonist-induced side effect.

### BACKGROUND OF THE INVENTION

Opioid agonists such as morphine, oxycodone, and hydrocodone are commonly prescribed to treat both acute and chronic pain, as their action on the opioid receptors can provide effective analgesia. However, the stimulating effect opioid agonists have on certain receptors may also cause an adverse pharmacodynamic response including bowel dysfunction that can be manifested by, e.g., decreased gastric motility, delayed gastric emptying, constipation, bloating and cramping. Other adverse pharmacodynamic responses associated with opioid therapy include nausea, vomiting, somnolence, dizziness, respiratory depression, headache, dry mouth, sedation, sweats, asthenia, hypotension, dysphoria, delirium, miosis, pruritis, urticaria, urinary retention, hyperalgesia, allodynia, physical dependence and tolerance.

Opioid-induced adverse pharmacodynamic responses in patients receiving opioid therapy for pain management can be particularly troublesome, as these patients are already trying to manage severe pain. The added discomfort of adverse side effects can add to their distress. In some cases, the side effects may be so extreme that the patient would rather discontinue use of the opioid than continue to suffer with such side effects.

Also, opioid agonist pharmaceutical dosage forms are sometimes the subject of abuse. For example, a particular dose of opioid agonist may be more potent when administered parenterally as compared to the same dose administered orally. Some formulations can be tampered with to provide the opioid agonist contained therein for illicit use. Controlled-release opioid agonist formulations are sometimes crushed or subject to extraction with solvents (e.g., ethanol) by drug abusers to provide the opioid contained therein for immediate release upon oral or parenteral administration.

There remains a need in the art for a composition and method to prevent or treat an opioid-induced adverse pharmacodynamic response. There also exists a need in the art for a composition and method to deter abuse of opioid analgesics.

All references cited herein are incorporated by reference in their entireties for all purposes.

### OBJECTS AND SUMMARY OF THE INVENTION

It is an object of certain embodiments of the present invention to provide a solid, controlled-release dosage form comprising an opioid antagonist which is abuse resistant.

It is an object of certain embodiments of the present invention to provide a solid, controlled-release dosage form comprising an opioid antagonist which is tamper resistant.

It is an object of certain embodiments of the present invention to provide a solid, controlled-release dosage form comprising an opioid analgesic and an opioid antagonist, which is resistant to crushing.

It is an object of certain embodiments of the present invention to provide a solid, controlled-release dosage form comprising an opioid antagonist and an opioid analgesic, which is subject to less opioid-induced adverse pharmacodynamic responses than other dosage forms.

It is an object of certain embodiments of the present invention to provide a solid, controlled-release dosage form comprising an opioid antagonist that targets release of at least a portion of the antagonist in the colon of a human subject.

It is an object of certain embodiments of the present invention to provide a solid, controlled-release dosage form comprising an opioid antagonist and opioid analgesic, which is subject to less parenteral abuse than other dosage forms.

It is an object of certain embodiments of the present invention to provide a solid, controlled-release dosage form comprising an opioid antagonist and an opioid analgesic, which is subject to less intranasal abuse than other dosage forms.

It is an object of certain embodiments of the present invention to provide a solid, controlled-release dosage form comprising an opioid antagonist and an opioid analgesic, which is subject to less oral abuse than other dosage forms.

It is a further object of certain embodiments of the present invention to provide a solid, controlled-release dosage form comprising an opioid antagonist and an opioid analgesic, which is subject to less diversion than other dosage forms.

It is a further object of certain embodiments of the present invention to provide a method of treating pain in human patients with a solid, controlled-release dosage form comprising an opioid analgesic and an opioid antagonist while reducing the abuse potential of the dosage form.

It is a further object of certain embodiments of the present invention to treat a disease or condition (e.g., pain) by administering a solid, controlled-release dosage form as disclosed herein to a patient in need thereof.

It is a further object of certain embodiments of the present invention to provide a method of manufacturing an oral dosage form of an active agent (e.g., an opioid analgesic) as disclosed herein.

It is a further object of certain embodiments of the present invention to provide a use of a medicament (e.g., an opioid analgesic) in the manufacture of a dosage form for the treatment of a disease state (e.g., pain).

It is another object of certain embodiments of the present invention to provide a use of a medicament (e.g., an opioid antagonist) in the preparation of any of the medicaments disclosed herein.

These objects and others are accomplished by the present invention, which in certain embodiments is directed to a solid, controlled-release dosage form comprising a core comprising a first portion of an opioid antagonist and a shell encasing the core and comprising a second portion of the opioid antagonist, wherein the release profile of the first portion of opioid antagonist is different than the release profile of the second opioid antagonist. In certain aspects of the invention, the dosage forms disclosed herein further comprise an opioid agonist.

In certain embodiments, the present invention is directed to a solid, controlled-release dosage form comprising a core comprising a first portion of an opioid antagonist and an antagonist-free shell encasing the core. In certain aspects of the invention, an opioid agonist is included in one of more of the core or the shell.

In certain embodiments, the invention is directed to a method of treating pain in a subject in need thereof, comprising administering to the subject a solid, controlled-release dosage form as disclosed herein.

In certain embodiments, the invention is directed to a method of preparing a solid controlled-release dosage form comprising preparing a core comprising a first portion of an opioid antagonist, and encasing the core with a shell comprising a second portion of the opioid antagonist, wherein the release profile of the first portion of opioid antagonist is different than the release profile of the second opioid antagonist.

In certain embodiments, the invention is directed to a method of preparing a solid, controlled-release dosage form comprising preparing a core comprising a first portion of an opioid antagonist dispersed in a first matrix material, and encasing the core with a shell comprising a second portion of the opioid antagonist dispersed in a second matrix material, wherein the release profile of the first portion of opioid antagonist is different than the release profile of the second opioid antagonist.

The term “zero-order release rate” refers to the rate of active agent released from a dosage form which is independent of the remaining active agent concentration in the dosage form, such that the rate is relatively constant over a period of time. A dosage form exhibiting zero order release rate would exhibit a relatively straight line in a graphical representation of percent active agent released versus time. In certain embodiments of the present invention, “substantial zero order release” is defined as a dosage form having an amount of active agent released which is proportional within 20% to elapsed time from about 8 to about 24 hours or about 4 to about 12 hours. Such active agent release is measured by an in-vitro dissolution in a USP Apparatus 1 (basket) at 100 rpm in 900 ml simulated gastric fluid without enzymes (SGF) at 37° C. For example, an amount released from a dosage form in-vitro at 8 hours of 20%, and an amount released at 24 hours of 60% (±12) would literally meet the definition of “proportional within 20% to elapsed time from about 8 to about 24 hours”. This is demonstrated by the latter elapsed time (24 hours) and the latter release (60%) being the same multiple (3) of the former time (8 hours) and the former release (20%). To meet the definition of “proportional within 20% to elapsed time from about 8 to about 24 hours” (or any other time period) it is only necessary to consider the endpoints of the numerical values, although the definition does not preclude that other time points within the endpoints may be proportional as well.

In other embodiments of the present invention, “substantial zero order release” is defined as a dosage form wherein the amount of active agent released at 2 hours is less than about 25%; the amount of active agent released from the dosage form at 4 hours is from about 10% to about 30%; the amount of active agent released from the dosage form at 8 hours is from about 20% to about 60%; the amount of active agent released from the dosage form at 12 hours is from about 40% to about 90%; and the amount of active agent released from the dosage form at 18 hours is greater than about 70%; as measured by an in-vitro dissolution in a USP Apparatus 1 (basket) at 100 rpm in 900 ml simulated gastric fluid without enzymes (SGF) at 37° C.

The term “polyethylene oxide” is defined for purposes of the present invention as a composition of polyethylene oxide (PEO) having a molecular weight of at least 25,000 daltons, measured as is conventional in the art based on rheological measurements, and preferably having a molecular weight of at least 100,000 daltons. Compositions with lower molecular weight are usually referred to as polyethylene glycol (PEG).

The term “high molecular weight polyethylene oxide (PEO)” is defined for proposes of the present invention as having an approximate molecular weight of at least 1,000,000 daltons, based on rheological measurements.

The term “low molecular weight polyethylene oxide (PEO)” is defined for purposes of the present invention as having an approximate molecular weight of less than 1,000,000 daltons, based on rheological measurements.

The term “direct compression” is defined for purposes of the present invention as referring to a process wherein the dosage form is made by a process comprising the steps of blending the ingredients and compressing the blend to form the dosage form, e.g., by using a diffusion blend and/or convection mixing process (e.g., Guidance for Industry, SUPAC-IR/MR: Immediate Release and Modified Release Solid Oral Dosage Forms, Manufacturing Equipment Addendum).

The term “flattening” and related terms, as used in the context of flattening a dosage form in accordance with the present invention, means that the dosage form is subjected to force applied from a direction substantially in line with the smallest diameter (i.e., the thickness) of the dosage form when the shape is other than spherical, and from any direction when the dosage form shape is spherical.

The term “resistant to crushing” is defined for the purposes of certain embodiments of the present invention as referring to dosage forms that can at least be flattened with a bench press as described herein without breaking.

For purposes of the present invention, the term “opioid analgesic” means one or more compounds selected from base opioid agonists, mixed opioid agonist-antagonists, partial opioid agonists, pharmaceutically acceptable salts, complexes, stereoisomers, ethers, esters, hydrates and solvates thereof and mixtures thereof.

The term “simulated gastric fluid” or “SGF” used herein refers to an aqueous solution utilized in dissolution testing to mimic the conditions of the stomach, e.g., a solution of 0.1 N HCl.

The term “percentage points” in the context of, e.g., “the amount of active agent released at 0.5 hour from a flattened dosage form deviates no more than about 20% points from a non-flattened dosage form” means that the difference in the % release prior to flattening and the % release after flattening is no more than 20% (i.e., 20% or less). For example, 60% release from a flattened dosage form is no more than about 20% points from the 40% release of a non-flattened dosage form.

The term “percentage” or the use of “%” without reference to “percentage (or %) points” is the ordinary meaning of percent. For example, 48% release is equal to and within 20% of 60% release, whereas 40% would not literally be within 20% of 60% release.

The term “patient” means a subject (preferably a human) who has presented a clinical manifestation of a particular symptom or symptoms suggesting the need for treatment, who is treated preventatively or prophylactically for a condition, or who has been diagnosed with a condition to be treated.

The term “subject” is inclusive of the definition of the term “patient” and inclusive of the term “healthy subject” (i.e., an individual (e.g., a human)) who is entirely normal in all respects or with respect to a particular condition.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached.

The term “enantiomer” or “enantiomeric” refers to a molecule that is not superimposable on its mirror image and hence optically active wherein the enantiomer rotates the plane of polarized light in one direction and its mirror image rotates the plane of polarized light in the opposite direction.

The term “racemic” refers to a mixture of enantiomers.

The term “resolution” refers to the separation, concentration or depletion of one of the two enantiomeric forms of a molecule.

“Hydrocodone” is defined for purposes of the invention as including hydrocodone free base, as well as pharmaceutically acceptable salts, complexes, stereoisomers, ethers, esters, hydrates and solvates thereof and mixtures thereof.

“Oxycodone” is defined for purposes of the invention as including oxycodone free base, as well as pharmaceutically acceptable salts, complexes, stereoisomers, ethers, esters, hydrates and solvates thereof and mixtures thereof.

“Naloxone” is defined for purposes of the invention as including naloxone free base, as well as pharmaceutically acceptable salts, complexes, stereoisomers, ethers, esters, hydrates and solvates thereof and mixtures thereof.

The term “USP Paddle or Basket Method” is the Paddle and Basket Method described, e.g., in U.S. Pharmacopoeia XII (1990).

The term “pH-dependent” for purposes of the present invention is defined as having characteristics (e.g., dissolution) which vary according to environmental pH.

The term “pH-independent” for purposes of the present invention is defined as having characteristics (e.g., dissolution) which are substantially unaffected by pH.

The term “bioavailability” is defined for purposes of the present invention as the relevant extent to which the drug (e.g., hydrocodone) is absorbed from the unit dosage forms. Bioavailability is also referred to as AUC (i.e., area under the plasma concentration/time curve).

The term “controlled-release”, “extended-release” or “sustained-release” are interchangeable and are defined for purposes of the present invention as the release of the drug (e.g., hydrocodone) at such a rate that blood (e.g., plasma) concentrations are maintained within the therapeutic range but below toxic concentrations over a period of time of at least about 12 hours or longer, or at least 24 hours or longer. Preferably, a controlled-release dosage form can provide once daily or twice daily dosing.

The term “Cₘₐₓ” denotes the maximum plasma concentration obtained during the dosing interval.

The term “C₂₄” as it is used herein is the plasma concentration of the drug at 24 hours after administration.

The term “Tₘₐₓ” denotes the time to maximum plasma concentration (Cₘₐₓ).

The term “C₂₄/Cₘₐₓ ratio” is defined for purposes of the present invention as the ratio of the plasma concentration of the drug at 24 hours after administration to the highest plasma concentration of the drug attained within the dosing interval.

The term “T_{lag}” denotes the time point immediately prior to the first measurable plasma concentration.

The term “T_{1/2}” denotes the terminal plasma half-life. This is the time it takes for any concentration in the terminal phase to decrease by half. The term “minimum effective analgesic concentration” or “MEAC” with respect to concentrations of opioids such as hydrocodone is very difficult to quantify. However, there is generally a minimally effective analgesic concentration of plasma hydrocodone below which no analgesia is provided. While there is an indirect relationship between, e.g., plasma hydrocodone levels and analgesia, higher and prolonged plasma levels are generally associated with superior pain relief. There is a delay (or hysteresis) between the time of peak plasma hydrocodone-levels and the time of peak drug effects. This holds true for the treatment of pain with opioid analgesics in general.

For purposes of the present invention, unless further specified, the term “a patient” or “a subject” means that the discussion (or claim) is directed to the pharmacokinetic parameters of an individual patient or subject.

The term “population of patients” or “population of subjects” or “population of healthy subjects” means that the discussion (or claim) is directed to the mean pharmacokinetic parameters of at least two patients, subjects, or healthy subjects; at least six patients, subjects or healthy subjects; or at least twelve patients, subjects or healthy subjects.

In certain embodiments, the controlled-release formulations disclosed herein are dose proportional. In dose proportional formulations, the pharmacokinetic parameters (e.g., AUC and Cₘₐₓ) and/or in-vitro release increase linearly from one dosage strength to another. Therefore, the pharmacokinetic and in-vitro parameters of a particular dose can be inferred from the parameters of a different dose of the same formulation.

The term “first administration” means a single dose of the present invention at the initiation of therapy to an individual subject, patient, or healthy subject or a subject population, patient population, or healthy subject population.

The term “steady state” means that the amount of the drug reaching the system is approximately the same as the amount of the drug leaving the system. Thus, at “steady-state”, the patient's body eliminates the drug at approximately the same rate that the drug becomes available to the patient's system through absorption into the blood stream.

### DETAILED DESCRIPTION

The present invention is directed to controlled-release pharmaceutical formulations that in certain embodiments comprise an opioid antagonist in an inner region of the dosage form and an opioid antagonist in an outer region of the dosage form. In certain embodiments, the inner and outer regions are configured as an inner core (e.g., a compressed tablet) and a shell encasing the core (e.g., a compression coating). In an alternative embodiment, the antagonist is contained solely in the core, without any antagonist in the shell. In preferred embodiments, at least a portion of the opioid antagonist is released in the colon in order to treat an opioid-induced adverse pharmacodynamics response (e.g., constipation).

In certain embodiments, the solid, controlled-release dosage form comprises a core comprising a first portion of an opioid antagonist and a shell encasing the core and comprising a second portion of the opioid antagonist, wherein the release profile of the first portion of opioid antagonist is different than the release profile of the second opioid antagonist.

In one embodiment, the release profile is defined by the amount of the opioid antagonist released from the core and the shell. For example, the amount of antagonist released from the core can be more than the amount of antagonist released from the shell. Alternatively, the amount of antagonist released from the core can be less than the amount of antagonist released from the shell.

In another embodiment, the release profile is defined by the release rate of the opioid antagonist. For example, the release rate of antagonist in the core can be higher than the release rate of antagonist in the shell. Alternatively, the release rate of antagonist in the core can be less than the release rate of antagonist in the shell.

In a further embodiment, the release profile is defined as the duration of release of the opioid antagonist. For example, the duration of release of antagonist from the core may be longer than the duration of release of antagonist from the shell. Alternatively, the duration of release of antagonist from the core can be less than the duration of release of antagonist from the shell. In a particular embodiment, the shell provides an immediate release of the antagonist and the core provides a controlled release of the antagonist. In one embodiment, the at least a portion of the antagonist in the core is not released until the dosage form enters the lower gastrointestinal tract (e.g., the colon).

The first portion and the second portion of opioid antagonist may be independently selected from the group consisting of, e.g., alvimopan, amiphenazole, cyclazacine, levallorphan, N-allyllevallorphan, naltrexone, methylnaltrexone, N-methylnaltrexone, naloxone, nalmefene, N-methylnalmefene, nadide, nalorphine, oxilorphan, pharmaceutically acceptable salts thereof and mixtures thereof. In a particular embodiment, the antagonist is naloxone hydrochloride. In another embodiment, the antagonist is naltrexone hydrochloride.

In one embodiment, the first portion of opioid antagonist comprises the same agent as the second portion of opioid antagonist. In other embodiments, the first portion of opioid antagonist comprises a different agent than the second portion of opioid antagonist. In a particular embodiment, the first portion of opioid antagonist and the second portion of opioid antagonist both comprise naloxone or a pharmaceutically acceptable salt thereof.

In one embodiment, the first portion of opioid antagonist is in an amount to deter the abuse of the dosage form. The abuse that is deterred may be, e.g., intravenous abuse or nasal abuse.

The dosage forms as disclosed herein may further comprise an opioid analgesic or be co-administered with an opioid analgesic. In such embodiments, the second portion of the opioid antagonist can be in an amount to inhibit an opioid-induced side effect (e.g., constipation). In certain embodiments, the antagonist in the core portion is released at least partially in the colon.

In certain embodiments containing opioid analgesics, the dosage forms of the present invention are tamper-resistant by being difficult to crush or grind (e.g., in accordance with the flattening criteria disclosed herein). This characteristic makes them especially suitable for controlled-release opioid analgesic products that have a large dose of opioid analgesic intended to be released over a period of time from each dosage unit. Drug abusers typically take a controlled-release product and crush, shear, grind, chew, dissolve, heat, extract or otherwise damage the product so that a large portion, or the full contents, of the opioid analgesic becomes available for immediate absorption by injection, inhalation, and/or oral consumption.

In certain embodiments, the shell of the dosage form of the present invention is difficult to physically separate from the core. This is particularly useful in embodiments that have an increased amount of opioid analgesic in the core as compared to the shell, as abusers will have difficulty in accessing the greater drug payload of the core.

In certain embodiments, the present invention is directed to a solid, controlled-release dosage form comprising a core comprising a first portion of an opioid antagonist dispersed in a first matrix material, and a shell encasing the core and comprising a shell portion of the opioid antagonist dispersed in a second matrix material.

The core of the dosage form can be formed, e.g., by direct compression, extrusion or molding. Preferably, the core comprises a controlled-release excipient and is in the form of a compressed tablet.

The shell of the dosage form can be formed, e.g., by compression coating, molding, spraying one or more layers onto the core, dipping one or more layers onto the core, or a combination thereof. Preferably, the shell comprises a controlled-release excipient and is a compression coating.

In preferred embodiments, the weight ratio of the core to the shell of the dosage forms described herein is from about 10:1 to about 1:10; from about 5:1 to about 1:5; from about 2:1 to about 1:5; from about 2:1 to about 1:2; from about 1:0.6 to about 1:1.5; or from about 1:0.8 to about 1:1.2.

In preferred embodiments, the core and the shell are visually indistinguishable from each other (e.g., by color) and there is not a clear demarcation between the two components. This contributes to tamper resistance of the dosage form by hindering efforts to isolate the core, which in certain embodiments will contain the bulk of the active agent. One measurement that can be utilized in order to evaluate the color of the shell and the core is CIE L*A*B* value. Preferably, the CIE L*A*B* value of the core and the shell are within 10% of each other. Another measurement to evaluate color is the use of a RYB or RGB color wheel, where the core and shell preferably correspond to the same hue or adjacent hues.

In certain embodiments, the first matrix material in the shell comprises polyethylene oxide (PEO). In other embodiments, the second matrix material in the core comprises PEO. In yet other embodiments, both the first matrix material in the shell and the second matrix material in the core comprise PEO. Preferably, PEO is contained in both components. In such embodiments, the average molecular weights of the PEO in the first matrix material is the same or different than the average molecular weight of the PEO in the second matrix material. In certain embodiments, the average molecular weights of the PEO contained in the core and the shell are within 20%, within 10% or within 5% of each other.

In preferred embodiments of the present invention, when PEO is present in both the first and second matrices, the average molecular weights of the PEO used in the first matrix in the core is lower than the average molecular weight of the PEO used in the second matrix material in the shell. For example, the PEO in the first matrix material may have an average molecular weight from about 300,000 daltons to about 10,000,000 daltons and the PEO in the second matrix material may have an average molecular weight from about 1,000,000 daltons to about 10,000,000 daltons. In other embodiments, the PEO in the first matrix material may have an average molecular weight from about 300,000 daltons to about 3,000,000 daltons and the PEO in the second matrix material may have an average molecular weight from about 4,000,000 daltons to about 10,000,000 daltons. In other embodiments, the PEO in the first matrix material may have an average molecular weight from about 500,000 daltons to about 1,000,000 daltons and the PEO in the second matrix material may have an average molecular weight from about 6,000,000 daltons to about 8,000,000 daltons.

In certain embodiments, the opioid antagonist in the core portion is the same as the opioid antagonist in the shell portion. In other embodiments, the opioid antagonist in the core portion is different than the opioid antagonist in the shell portion.

In certain embodiments containing an opioid analgesic, the weight ratio of opioid analgesic in the core to the ratio of opioid analgesic in the shell is from about 1:1 to about 10:1; from about 2:1 to about 8:1; from about 2:1 to about 5:1 or about 4:1.

In certain embodiments, the weight ratio of a first portion of opioid analgesic in the core to PEO in the first matrix material is from about 4:1 to about 1:30; from about 2:1 to about 1:100; from about 2:1 to about 1:20; from about 1:1 to about 1:10; from about 1:15 to about 1:20; from about 1:1.5 to about 1:4; or about 1:2.

In alternative embodiments, the weight ratio of a second portion of opioid analgesic in the shell to PEO in the second matrix material is from about 1:1 to about 1:200; from about 1:1 to about 1:125; from about 1:2 to about 1:100; from about 1:5 to about 1:50; from about 1:12 to about 1:25, about 1:98 or about 1:15.

In certain embodiments containing an opioid analgesic, the amount of opioid analgesic released from the dosage form is proportional within 20%, or within 10%, or within 5% to elapsed time from 8 to 24 hours, as measured by an in-vitro dissolution in a USP Apparatus 1 (basket) at 100 rpm in 900 ml simulated gastric fluid without enzymes (SGF) at 37° C.

In certain embodiments containing an opioid analgesic, the amount of opioid analgesic released from the dosage form is proportional within 20%, or within 10%, or within 5% to elapsed time from 8 to 18 hours, as measured by an in-vitro dissolution in a USP Apparatus 1 (basket) at 100 rpm in 900 ml simulated gastric fluid without enzymes (SGF) at 37° C.

In certain embodiments containing an opioid analgesic, the amount of opioid analgesic released from the dosage form is proportional within 20%, or within 10%, or within 5% to elapsed time from 8 to 12 hours, as measured by an in-vitro dissolution in a USP Apparatus 1 (basket) at 100 rpm in 900 ml simulated gastric fluid without enzymes (SGF) at 37° C.

In certain embodiments containing an opioid analgesic, the amount of opioid analgesic released from the dosage form is proportional within 20%, or within 10%, or within 5% to elapsed time from 12 to 24 hours, as measured by an in-vitro dissolution in a USP Apparatus 1 (basket) at 100 rpm in 900 ml simulated gastric fluid without enzymes (SGF) at 37° C.

In certain embodiments containing an opioid analgesic, the amount of opioid analgesic released from the dosage form is proportional within 20%, or within 10%, or within 5% to elapsed time from 12 to 18 hours, as measured by an in-vitro dissolution in a USP Apparatus 1 (basket) at 100 rpm in 900 ml simulated gastric fluid without enzymes (SGF) at 37° C.

In certain embodiments containing an opioid analgesic, the amount of opioid analgesic released from the dosage form is proportional within 20%, or within 10%, or within 5% to elapsed time from 4 to 20 hours, as measured by an in-vitro dissolution in a USP Apparatus 1 (basket) at 100 rpm in 900 ml simulated gastric fluid without enzymes (SGF) at 37° C.

In certain embodiments containing an opioid analgesic, the amount of opioid analgesic released from the dosage form is proportional within 20%, or within 10%, or within 5% to elapsed time from 4 to 15 hours, as measured by an in-vitro dissolution in a USP Apparatus 1 (basket) at 100 rpm in 900 ml simulated gastric fluid without enzymes (SGF) at 37° C.

In certain embodiments containing an opioid analgesic, the amount of opioid analgesic released from the dosage form is proportional within 20%, or within 10%, or within 5% to elapsed time from 4 to 10 hours, as measured by an in-vitro dissolution in a USP Apparatus 1 (basket) at 100 rpm in 900 ml simulated gastric fluid without enzymes (SGF) at 37° C.

In certain embodiments containing an opioid analgesic, the amount of opioid analgesic released from the dosage form is proportional within 20%, or within 10%, or within 5% to elapsed time from 8 to 20 hours, as measured by an in-vitro dissolution in a USP Apparatus 1 (basket) at 100 rpm in 900 ml simulated gastric fluid without enzymes (SGF) at 37° C.

In certain embodiments containing an opioid analgesic, the amount of opioid analgesic released from the dosage form is proportional within 20%, or within 10%, or within 5% to elapsed time from 10 to 15 hours, as measured by an in-vitro dissolution in a USP Apparatus 1 (basket) at 100 rpm in 900 ml simulated gastric fluid without enzymes (SGF) at 37° C.

In certain embodiments containing an opioid analgesic, the amount (by weight) of opioid analgesic released at 2 hours is less than about 25%; the amount of opioid released from the dosage form at 4 hours is from about 10% to about 30%; the amount of opioid released from the dosage form at 8 hours is from about 20% to about 60%; the amount of opioid released from the dosage form at 12 hours is from about 40% to about 90%; and the amount of opioid released from the dosage form at 18 hours is greater than about 70%; as measured by an in-vitro dissolution in a USP Apparatus 1 (basket) at 100 rpm in 900 ml simulated gastric fluid without enzymes (SGF) at 37° C.

In certain embodiments containing an opioid analgesic, the amount (by weight) of opioid analgesic released at 2 hours is less than about 15%; the amount of opioid released from the dosage form at 4 hours is from about 10% to about 20%; the amount of opioid released from the dosage form at 8 hours is from about 30% to about 45%; the amount of opioid released from the dosage form at 12 hours is from about 50% to about 70%; and the amount of opioid released from the dosage form at 18 hours is greater than about 90%; as measured by an in-vitro dissolution in a USP Apparatus 1 (basket) at 100 rpm in 900 ml simulated gastric fluid without enzymes (SGF) at 37° C.

In certain embodiments containing an opioid analgesic, the amount (by weight) of opioid analgesic released at 2 hours is less than about 10%; the amount of opioid released from the dosage form at 4 hours is from about 20% to about 30%; the amount of opioid released from the dosage form at 8 hours is from about 45% to about 60%; the amount of opioid released from the dosage form at 12 hours is from about 70% to about 90%; and the amount of opioid released from the dosage form at 18 hours is greater than about 95%; as measured by an in-vitro dissolution in a USP Apparatus 1 (basket) at 100 rpm in 900 ml simulated gastric fluid without enzymes (SGF) at 37° C.

In certain embodiments containing an opioid analgesic, the amount (by weight) of opioid analgesic released from the dosage form is proportional within 20% to elapsed time from 8 to 24 hours, as measured by an in-vitro dissolution in a USP Apparatus 1 (basket) at 100 rpm in 900 ml simulated gastric fluid without enzymes (SGF) at 37° C. and at least one of the following is exhibited: (i) the amount of opioid analgesic released at 2 hours is less than about 20%, (ii) the amount of opioid analgesic released at 4 hours is from about 10% to about 30%, (iii) the amount of opioid analgesic released at 8 hours is from about 30% to about 60%, (iv) the amount of opioid analgesic released at 12 hours is from about 50% to about 90%, or (v) the amount of opioid analgesic released at 18 hours is greater than about 80%.

In certain embodiments containing an opioid analgesic, the amount (by weight) of opioid analgesic released from the dosage form is proportional within 20% to elapsed time from 8 to 24 hours, as measured by an in-vitro dissolution in a USP Apparatus 1 (basket) at 100 rpm in 900 ml simulated gastric fluid without enzymes (SGF) at 37° C. and at least one of the following is exhibited: (i) the amount of opioid analgesic released at 2 hours is less than about 15%, (ii) the amount of opioid analgesic released at 4 hours is from about 10% to about 20%, (iii) the amount of opioid analgesic released at 8 hours is from about 30% to about 45%, (iv) the amount of opioid analgesic released at 12 hours is from about 50% to about 70%, or (v) the amount of opioid analgesic released at 18 hours is greater than about 90%.

In certain embodiments containing an opioid analgesic, the amount (by weight) of opioid analgesic released from the dosage form is proportional within 20% to elapsed time from 8 to 24 hours, as measured by an in-vitro dissolution in a USP Apparatus 1 (basket) at 100 rpm in 900 ml simulated gastric fluid without enzymes (SGF) at 37° C. and at least one of the following is exhibited: (i) the amount of opioid analgesic released at 2 hours is less than about 10%, (ii) the amount of opioid analgesic released at 4 hours is from about 20% to about 30%, (iii) the amount of opioid analgesic released at 8 hours is from about 45% to about 60%, (iv) the amount of opioid analgesic released at 12 hours is from about 70% to about 90%, or (v) the amount of opioid analgesic released at 18 hours is greater than about 95%.

In certain embodiments containing an opioid analgesic, the amount of opioid analgesic (by weight) released from the dosage form is proportional within 20% to elapsed time from 8 to 24 hours, as measured by an in-vitro dissolution in a USP Apparatus 1 (basket) at 100 rpm in 900 ml simulated gastric fluid without enzymes (SGF) at 37° C. and at least one of the following is exhibited: (i) the amount of opioid analgesic released at 2 hours is less than about 15%, (ii) the amount of opioid analgesic released at 4 hours is from about 8% to about 20%, (iii) the amount of opioid analgesic released at 8 hours is from about 20% to about 50%, (iv) the amount of opioid analgesic released at 12 hours is from about 40% to about 70%, (v) the amount of opioid analgesic released at 18 hours is greater than about 70% or (vi) the amount of opioid analgesic released from the dosage form at 24 hours is greater than about 90%.

### Dosage Forms

In certain embodiments, the core may be prepared by dry blending a controlled-release matrix material, an opioid antagonist, an optional opioid agonist and optionally various excipients, followed by granulating the mixture until proper granulation is obtained. The process can be performed by dry or wet granulation methods. Typically with a wet granulation, the wet granules are dried in a fluid bed dryer, and sifted and ground to appropriate size. Lubricating agents are typically mixed with the granulation to obtain the final core formulation.

Pharmaceutical compositions of the invention include single unit dosage forms suitable for oral administration such as, but not limited to, tablets, capsules, gelcaps, and caplets, which may be adapted for controlled or immediate release.

In some embodiments, the controlled-release component may include gels, permeable membranes, osmotic systems, multilayer coatings, microparticles, multiparticulates, liposomes, microspheres, or a combination thereof to provide the desired release profile in varying proportions.

A non-limiting list of suitable controlled-release matrix materials which may be selected for inclusion in a formulation according to the present invention includes hydrophilic and hydrophobic materials such as sustained release polymers, gums, acrylic resins, protein-derived materials, waxes, shellacs, and oils such as hydrogenated castor oil and hydrogenated vegetable oil. More specifically, the controlled-release materials can be, e.g., polyalkylene oxides such as polyethylene oxide (PEO), alkylcelluloses such as ethylcellulose, acrylic and methacrylic acid polymers and copolymers, and cellulose ethers such as hydroxyalkylcelluloses (e.g., hydroxypropylmethylcellulose, HPMC) and carboxyalkylcelluloses. Waxes include, e.g., natural and synthetic waxes, fatty acids, fatty alcohols, and mixtures of the same (e.g., beeswax, carnauba wax, stearic acid and stearyl alcohol). Certain embodiments utilize mixtures of two or more of the foregoing controlled-release materials in the matrix of the core. However, any pharmaceutically acceptable hydrophobic or hydrophilic controlled-release material capable of imparting controlled-release of the active agent may be used in accordance with the present invention.

In other embodiments, controlled-release polymers may include methyl methacrylate, methyl methacrylate copolymers, ethoxyethyl methacrylates, ethyl acrylate, trimethyl ammonioethyl methacrylate, cyanoethyl methacrylate, aminoalkyl methacrylate copolymer, poly(acrylic acid), poly(methacrylic acid), methacrylic acid alkylamine copolymer, methacrylic acid and ethyl acrylate copolymers, vinyl acetate and vinyl pyrrolidone copolymers, vinyl acetate and crotonic acid copolymers, polymethyl vinyl ether and malonic acid anhydride copolymers, polymethyl vinyl ether and malonic acid or the ethyl-, isopropyl-, n-butylesters thereof copolymers, poly(methylmethacrylate), poly(methacrylicacid) (anhydride), polymethacrylate, polyacrylamide, poly(methacrylic acid anhydride), acetyl succinate, polyvinyl acetate, polyvinyl acetate phthalate, polyvinylpyrollidone, polyacrylic acid, polysaccharides, modified starch, cross-linked high amylose starch, hydroxypropyl starch, starch, hydroxypropyl methylcellulose phthalate, microcrystalline cellulose, carboxymethylethyl cellulose, cellulose acetate, methylcellulose, ethyl cellulose, hydroxypropyl cellulose, cellulose phthalate, cellulose acetate, cellulose acetate phthalate, cellulose acetate propionate, cellulose acetate succinate, cellulose acetate butyrate, cellulose acetate trimellitate, poloxamer, povidone, alginic acid, sodium alginate, polyethylene glycol alginate, xanthan gum, polymethacrylates, zein, and glycidyl methacrylate copolymers. Certain preferred embodiments utilize mixtures of any of the foregoing sustained-release materials in the matrix of the invention.

The core may also contain suitable quantities of additional excipients, e.g., lubricants, binders, granulating aids, diluents, colorants, flavorants (e.g., irritants or bittering agents) and glidants, all of which are conventional in the pharmaceutical art.

Specific examples of pharmaceutically acceptable diluents and excipients that may be used in formulating the core are described in the Handbook of Pharmaceutical Excipients, American Pharmaceutical Association (1986), incorporated by reference herein in view of this disclosure.

In preferred embodiments, matrices of the dosage forms of the present invention incorporate PEO (e.g., high and/or low molecular weight PEO).

PEO is considered to have an average molecular weight of 1,000,000 daltons when a 2% (by wt) aqueous solution of the PEO using a Brookfield viscometer Model RVF, spindle No. 1, at 10 rpm, at 25° C. shows a viscosity range of 400 to 800 mPa-s (cP).

PEO is considered to have an average molecular weight of 2,000,000 daltons when a 2% (by wt) aqueous solution of the PEO using a Brookfield viscometer Model RVF, spindle No. 3, at 10 rpm, at 25° C. shows a viscosity range of 2000 to 4000 mPa-s (cP).

PEO is considered to have an average molecular weight of 4,000,000 daltons when a 1% (by wt) aqueous solution of the PEO using a Brookfield viscometer Model RVF, spindle No. 2, at 2 rpm, at 25° C. shows a viscosity range of 1650 to less than 5500 mPa-s (cP).

PEO is considered to have an average molecular weight of 5,000,000 daltons when a 1% (by wt) aqueous solution of the PEO using a Brookfield viscometer Model RVF, spindle No. 2, at 2 rpm, at 25° C. shows a viscosity range of 5500 to less than 7500 mPa-s (cP).

PEO is considered to have an average molecular weight of 7,000,000 daltons when a 1% (by wt) aqueous solution of the PEO using a Brookfield viscometer Model RVF, spindle No. 2, at 2 rpm, at 25° C. shows a viscosity range of 7500 to less than 10,000 mPa-s (cP).

PEO is considered to have an average molecular weight of 8,000,000 daltons when a 1% (by wt) aqueous solution of the PEO using a Brookfield viscometer Model RVF, spindle No. 2, at 2 rpm, at 25° C. shows a viscosity range of 10,000 to 15,000 mPa-s (cP).

Regarding the lower molecular weight PEOs, PEO is considered to have an average molecular weight of 100,000 daltons when a 5% (by wt) aqueous solution of the polyethylene oxide using a Brookfield viscometer Model RVT, spindle No. 1, at 50 rpm, at 25° C. shows a viscosity range of 30 to 50 mPa-s (cP).

Polyethylene oxide is considered to have an average molecular weight of 900,000 daltons when a 5% (by wt) aqueous solution of the PEO using a Brookfield viscometer Model RVF, spindle No. 2, at 2 rpm, at 25° C. shows a viscosity range of 8800 to 17,600 mPa-s (cP).

### Compression Coated Dosage Forms

In embodiments utilizing compression coating to create the shell, it is preferred that all or part of the pharmaceutically acceptable excipient(s) in the coating should impart sufficient compressibility to provide a pharmaceutically acceptable product. The compression coating of the shell onto the pre-formed core is dependent in part on the individual characteristics of the selected excipients and the active agent, e.g., in terms of polymer solubility, flowability, glass transition temperature, etc.

In non-limiting embodiments, compression coated dosage forms can be prepared, e.g., by utilizing a pre-manufactured core or preparing a core (e.g., by compression) prior to the coating. The inner core can be prepared by wet or dry granulating an opioid antagonist (with or without an opioid agonist) together with the pharmaceutically acceptable excipients; followed by drying and milling as necessary to obtain a granulate; adding optional extragranular excipients and/or opioid antagonist or optional opioid agonist with appropriate blending; adding a lubricant as needed; and compressing the granulate with a tablet press. The resultant compressed core can be optionally coated with a functional coating or film coating prior to compression coating.

The blend for compression coating of the shell can be prepared by a similar process as the blend for the core utilizing any of the controlled-release materials disclosed above. Preferably, the compression coating includes PEO. The blend can be coated onto the core by compression. The compression of the core and/or the coating can utilize a Killion or Fette rotary press at a compression force, e.g., from about 1 to about 20 kilo-newtons.

In certain embodiments, a Manesty Dry-Cota press (e.g., Model 900) can be utilized. This apparatus consists of two side-by-side interconnected tablet presses where the core is made on one press and then mechanically transferred to the next press for compression coating. Each press has an independent powder feed mechanism so that the core blend is loaded on one machine, and the coating blend is loaded on the other machine. Mechanical transfer arms rotate between the machines to remove cores from the core press and transfer them to the coating press. Other presses which may be used to prepare the dosage forms of the present invention include Elizabeth Hata HT-AP44-MSU-C; Killian RLUD; and Fette PT 4090, each of which has a dual feed system for coating blend and pre-made cores. Utilizing these presses allows multiple compression coating-layers to be achieved by recycling tablets that have already been compression-coated. All of these presses have mechanisms to center the tablet within the coating blend both vertically and radially.

In certain embodiments, the shell is not applied at the same thickness at all points around the core, but instead is applied at different thicknesses around the inner core. Thinner areas of coating will produce areas of the dosage form that will release the drug from the core sooner than other areas. This may be simply accomplished, e.g., by having the core to which the shell is being applied not being centered in the press at the time of coating.

In certain embodiments, the shell can be further over-coated with a hydrophobic or enteric coating material. In other embodiments, the shell can be coated with a hydrophilic coating in addition to, or instead of, the hydrophobic or enteric coating.

In still further embodiments, an optional coat (e.g., hydrophobic, hydrophilic or enteric) may be alternatively or additionally applied as an intermediate layer between the core and the shell.

### Active Agents

In addition to the opioid antagonist, certain embodiments include an opioid analgesic in the core, in the shell, or in both components. Opioid analgesics useful in the present invention include, but are not limited to, alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl and derivatives, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tramadol, pharmaceutically acceptable salts, complexes (e.g., with a cyclodextrin), stereoisomers, ethers, esters, hydrates, solvates, derivatives and mixtures thereof.

Preferably, the opioid analgesic is selected from the group consisting of codeine, hydrocodone, hydromorphone, morphine, oxycodone, oxymorphone, tramadol, pharmaceutically acceptable salts, complexes, stereoisomers, ethers, esters, hydrates, solvates, derivatives and mixtures thereof.

In certain embodiments, the opioid analgesic is selected from the group consisting of hydrocodone, pharmaceutically acceptable salts, derivatives and mixtures thereof. In one embodiment, the opioid analgesic is hydrocodone bitartrate.

In other embodiments, the opioid analgesic is selected from the group consisting of oxycodone, pharmaceutically acceptable salts, derivatives and mixtures thereof. In one embodiment, the opioid analgesic is oxycodone hydrochloride.

The opioid antagonists or agonists used according to the present invention may contain one or more asymmetric centers and may give rise to enantiomers, diastereomers, or other stereoisomeric forms. The present invention is meant to encompass the use of all such possible forms as well as their racemic and resolved forms and compositions thereof. When the compounds described herein contain olefinic double bonds or other centers of geometric asymmetry, it is intended to include both E and Z geometric isomers. All tautomers are intended to be encompassed by the present invention as well.

Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparaginate, glutamate and the like; metal salts such as sodium salt, potassium salt, cesium salt and the like; alkaline earth metals such as calcium salt, magnesium salt and the like; and organic amine salts such as triethylamine salt, pyridine salt, picoline salt, ethanolamine salt, triethanolamine salt, dicyclohexylamine salt, or N,N′-dibenzylethylenediamine salt and the like.

In certain embodiments, the formulations disclosed herein can be utilized for delivery of an active agent other than an opioid antagonist to the colon of a patient. The drug can be a peptide prone to degradation in the stomach and/or small intestine. The drug can also be a therapeutic agent for the treatment of diabetes mellitus, osteoporosis, neurodegenerating diseases, endometriosis, acromegaly, diabetes insipidus, colorectal cancer, ulcerative colitis, immune abnormality diseases, inflammatory bowel disease, Crohn's disease, irritable colon syndrome. Specific classes of drugs include an antitumor agent, antibiotic, polypeptide, anti-inflammatory agent, chemotherapeutic agent, immunosuppressant, steroid agent, vitamin, laxative, polynucleotide or Lactobacillus pharmaceutical preparation.

Examples of the specific agents include human insulin, peptide with human insulin-like action, thyroid hormone (PTH), peptide with PTH-like action, human calcitonin, peptide with human calcitonin-like action, thyrotropin releasing hormone (TRH), taltirelin hydrate, luteinizing hormone-releasing hormone (LH-RH), goserelin acetate, buserelin acetate, nafarelin acetate, oxitocin, human hypophyseal gonadotropin, octreotide acetate, somatropin, human chorionic gonadotropin, desmopressin acetate, 5-fluorouracil, bleomycin, doxifluridine, tegafur, tegafur 5-aminosalicylic acid, salazosulfapyridine, infliximab, budesonide, fluticasone propionate, beclometasone propionate ester, dexamethasone, dexamethasone acetate, dexamethasone palmitate, dexamethasone sodium metasulfobenzoate, dexamethasone sodium phosphate, triamcinolone, triamcinolone acetonide, hydrocortisone, hydrocortisone succinic acid ester sodium, fludrocortisone acetate, prednisolone, prednisolone succinic acid ester sodium, prednisolone sodium phosphate, beclometasone propionate ester, betamethasone, betamethasone d-chlorpheniramine maleic acid, betamethasone acetate, betamethasone sodium phosphate ester, betamethasone sodium phosphate ester, methylprednisolone, methylprednisolone sodium succinate or methylprednisolone acetate.

In yet further embodiments, the dosage may further include a non-opioid drug. Such non-opioid drugs include, but are not limited to, aspirin, acetaminophen; non-steroidal anti-inflammatory drugs (“NSAIDS”), e.g., ibuprofen, ketoprofen, etc.; N-methyl-D-aspartate (NMDA) receptor antagonists, e.g., a morphinan such as dextromethorphan or dextrorphan, or ketamine; cyclooxygenase-II inhibitors (“COX-II inhibitors”); and/or glycine receptor antagonists.

In certain embodiments of the present invention comprising an opioid analgesic, the invention allows for the use of lower doses of the opioid analgesic by virtue of the inclusion of additional non-opioid analgesics. By using lower amounts of opioid analgesics, the side effects associated with opioids in humans are also reduced.

Suitable non-steroidal anti-inflammatory agents, including ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidornetacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam or isoxicam, and the like, including all pharmaceutically acceptable salts thereof. Useful dosages of these drugs are well known to those skilled in the art.

N-methyl-D-aspartate (NMDA) receptor antagonists are well known in the art, and encompass, for example, morphinans such as dextromethorphan or dextrorphan, ketamine, or pharmaceutically acceptable salts thereof. For purposes of the present invention, the term “NMDA antagonist” is also deemed to encompass drugs that block a major intracellular consequence of NMDA-receptor activation, e.g. a ganglioside such as GM₁ or GT_{1b} a phenothiazine such as trifluoperazine or a naphthalenesulfonamide such as N-(6-aminohexyl)-5-chloro-1-naphthalene sulfonamide.

The COX-II inhibitors may be selected from the group consisting of celecoxib, rofecoxib, valdecoxib, parecoxib, lumiracoxib, etoricoxib, firocoxib, flosulide, meloxicam, nabumetone, nimesulide and pharmaceutically acceptable salts thereof. In yet further embodiments, a non-opioid drug can be included which provides a desired effect other than analgesia, e.g., antitussive, expectorant, decongestant, antihistamine drugs, local anesthetics, and the like.

### Antagonist Formulations

In certain embodiments of the present invention, the total amount of (e.g., naloxone or a pharmaceutically acceptable salt thereof or naltrexone or a pharmaceutically acceptable salt thereof). The may be, e.g., from about 0.01 mg to about 100 mg; from about 0.1 mg to about 50 mg; from about 0.1 mg to about 25 mg; from about 0.5 mg to about 15 mg or from about 1 mg to about 12 mg.

The amount of antagonist included in the shell (e.g., naloxone or a pharmaceutically acceptable salt thereof or naltrexone or a pharmaceutically acceptable salt thereof) is preferably an amount that deters intravenous or nasal abuse where the dosage form also contains an opioid analgesic. In such an embodiment, naloxone or a pharmaceutically acceptable salt thereof may be present in an amount, e.g., from about 0.1 mg to about 5 mg; from about 0.5 mg to about 1 mg; about 0.5 mg or about 0.85 mg.

The amount of antagonist included in the core is preferably an amount that treats an opioid induced adverse pharmacodynamics effect such as constipation. In such an embodiment, naloxone or a pharmaceutically acceptable salt thereof may be present in an amount, e.g., from about 0.1 mg to about 5 mg; from about 0.5 mg to about 1 mg; or from about 0.5 mg to about 0.85 mg.

### Oxycodone/Antagonist Embodiments

In addition to the opioid antagonist, the controlled-release oral dosage forms of the present invention may include, e.g., from about 2.5 mg to about 320 mg oxycodone or an equivalent amount of a pharmaceutically acceptable salt thereof. In other embodiments, the dosage forms contain from about 5 mg to about 240 mg oxycodone or an equivalent amount of a pharmaceutically acceptable salt thereof, or from about 5 mg to about 120 mg oxycodone or an equivalent amount of a pharmaceutically acceptable salt thereof. In certain embodiments, the opioid agonist is oxycodone or a pharmaceutically acceptable salt thereof (e.g., oxycodone hydrochloride) in an amount, e.g., of about 2.5 mg, 5 mg, 7.5 mg, 10 mg, 15 mg, 20 mg, 25 mg, 30 mg, 40 mg, 60 mg, 80 mg, 120 mg, 160 mg or 320 mg.

In certain embodiments of the present invention, wherein the active agent is oxycodone hydrochloride, the oxycodone hydrochloride has a 14-hydroxycodeinone level of less than about 25 ppm, less than about 15 ppm, less than about 10 ppm, less than about 5 ppm, less than about 2 ppm, less than about 1 ppm, less than about 0.5 ppm or less than about 0.25 ppm.

WO 2005/097801 A1, U.S. Pat. No. 7,129,248 B2 and US 2006/0173029 A1, all of which are hereby incorporated by reference, describe a process for preparing oxycodone hydrochloride having reduced levels of 14-hydroxycodeinone

### Hydrocodone/Antagonist Embodiments

In addition to the opioid antagonist, the controlled-release oral dosage forms of the present invention may include, e.g., from about 0.5 mg to about 1250 mg hydrocodone or an equivalent amount of a pharmaceutically acceptable salt thereof. In other embodiments, the dosage forms contain from about 2 mg to about 200 mg hydrocodone or an equivalent amount of a pharmaceutically acceptable salt thereof, or from about 16 mg to about 120 mg hydrocodone or an equivalent amount of a pharmaceutically acceptable salt thereof. In certain preferred embodiments, the dosage form contains about 20 mg, about 30 mg, about 40 mg, about 60 mg, about 80 mg, about 100 mg or about 120 mg hydrocodone bitartrate.

Suitable pharmaceutically acceptable salts of hydrocodone include hydrocodone bitartrate, hydrocodone bitartrate hydrate, hydrocodone hydrochloride, hydrocodone p-toluenesulfonate, hydrocodone phosphate, hydrocodone thiosemicarbazone, hydrocodone sulfate, hydrocodone trifluoroacetate, hydrocodone hemipentahydrate, hydrocodone pentafluoropropionate, hydrocodone p-nitrophenylhydrazone, hydrocodone o-methyloxime, hydrocodone semicarbazone, hydrocodone hydrobromide, hydrocodone mucate, hydrocodone oleate, hydrocodone phosphate dibasic, hydrocodone phosphate monobasic, hydrocodone inorganic salt, hydrocodone organic salt, hydrocodone acetate trihydrate, hydrocodone bis(heptafuorobutyrate), hydrocodone bis(methylcarbamate), hydrocodone bis(pentafluoropropionate), hydrocodone bis(pyridine carboxylate), hydrocodone bis(trifluoroacetate), hydrocodone chlorhydrate, and hydrocodone sulfate pentahydrate. Preferably, the hydrocodone is present as the bitartrate salt.

In preferred embodiments, the hydrocodone formulations of the present invention are suitable for once-a-day administration and provide a relatively flat plasma profile, meaning that the plasma level of the hydrocodone provides a C₂₄/Cₘₐₓ ratio of about 0.55 to about 1.0 after administration. In certain embodiments, the C₂₄/Cₘₐₓ ratio is about 0.55 to about 0.85, about 0.55 to about 0.75 or about 0.60 to about 0.70 after single dose administration of the dosage form or at steady state. Other embodiments are suitable for twice-a-day administration.

In preferred embodiments, the hydrocodone formulations of the present invention provide a Tₘₐₓ (h) of hydrocodone from about 4 to about 20 hours after administration. In certain embodiments, the Tₘₐₓ is about 6 to about 12 hours, about 8 to about 10 hours, about 4 to about 10 hours, about 8 to about 14 hours, or about 14 to about 20 hours after administration of the dosage form.

In still other embodiments, a solid, controlled-release dosage form of the present invention provides an AUC (ng*h/mL) after administration of about 200 to 450 or about 250 to 400 per each 20 mg hydrocodone or pharmaceutically acceptable salt thereof included in the dosage form.

In certain embodiments, a solid, controlled-release dosage form that contains 20 mg hydrocodone or a pharmaceutically acceptable salt thereof provides an AUC (ng*h/mL) after administration of about 200 to about 450, about 250 to about 400, about 275 to about 350, about 300 to 330 or about 280 to about 320.

In certain embodiments, a solid, controlled-release dosage form that contains 120 mg hydrocodone or a pharmaceutically acceptable salt thereof provides an AUC (ng*h/mL) after administration of about 1000 to about 3000, about 1500 to about 2400, about 1700 to about 2200, about 1800 to about 2100 or about 1900 to about 2100.

In other embodiments, a solid, controlled-release dosage form of the present invention provides a Cₘₐₓ (ng/mL) after administration of about 5 to about 40, about 10 to about 30 per each 20 mg hydrocodone included in the dosage form.

In certain embodiments, a solid, controlled-release dosage form that contains 20 mg hydrocodone or a pharmaceutically acceptable salt thereof provides a Cₘₐₓ (ng/mL) after administration of about 5 to about 40, about 10 to about 30, about 12 to about 25, about 14 to about 18 or about 12 to about 17.

In certain embodiments, a solid, controlled-release dosage form that contains 120 mg hydrocodone or a pharmaceutically acceptable salt thereof provides a Cₘₐₓ (ng/mL) after administration of about 30 to about 120, about 60 to about 180, about 100 to about 160, about 110 to about 150 or about 100 to about 140.

In certain embodiments, a solid, controlled-release dosage form of the present invention provides a Tₘₐₓ (h) of hydrocodone after administration of about 7 to about 22, about 10 to about 20, about 12 to about 18, about 13 to about 17 or about 14 to about 16.

In other embodiments, a solid, controlled-release dosage form of the present invention provides a T_{1/2} (h) of hydrocodone after administration of about 5 to about 10; about 6 to about 9 or about 7 to about 8.

In other embodiments, a solid, controlled-release dosage form of the present invention provides a T_{lag} (h) of hydrocodone after administration of about 0.01 to about 0.2; about 0.1 to about 0.18; about 0.3 to about 0.17; or about 0.06 to about 0.15.

In other embodiments, a solid, controlled-release dosage form of the present invention provides a C₂₄/Cₘₐₓ ratio of hydrocodone of about 0.2 to about 0.8; about 0.3 to about 0.7; or about 0.4 to about 0.6.

In certain embodiments, the above in vivo parameters are mean values. In other embodiments the in vivo parameters are achieved after administration in the fasted state.

In certain embodiments, the mean AUC (ng*h/mL) of hydrocodone after administration in the fed state is less than 20% higher, less than 16% higher or less than 12% higher than the AUC (ng*h/mL) of the same dose of hydrocodone after administration in the fasted state.

In certain embodiments, the mean Cₘₐₓ (ng/mL) of hydrocodone after administration in the fed state is less than 80% higher, less than 70% higher or less than 60% higher than the Cₘₐₓ of hydrocodone after administration of the same dose of hydrocodone in the fasted state.

In certain embodiments, the mean Tₘₐₓ (h) of hydrocodone after administration in the fed state is within 25%, within 20% or within 15% of the Tₘₐₓ of hydrocodone after administration of the same dose of hydrocodone in the fasted state.

In certain embodiments, the mean T_{1/2} (h) of hydrocodone after administration in the fed state is within 8%, within 5% or within 2% of the T_{1/2} after administration of the same dose of hydrocodone in the fasted state.

In certain embodiments, the mean T_{lag} of hydrocodone after administration in the fed state is less than 150% higher, less than 125% higher or less than 100% higher than the T_{1/2} after administration of the same dose of hydrocodone in the fasted state.

In certain embodiments, any one or all of the above in vivo parameters are achieved after a first administration of the dosage form to a human subject, patient or healthy subject (individual data) or a population of human subjects, patients or healthy subjects (mean data).

In certain alternative embodiments, any one or all of the above in vivo parameters are achieved after steady state administration of the dosage form to a human subject, patient or healthy subject or a population of human subjects, patients or healthy subjects.

### Cured Formulations

In certain embodiments, a process of the present invention further comprises the step of curing the final dosage form.

For embodiments comprising polyethylene oxide in a controlled-release formulation, the curing step may comprise at least partially melting the polyethylene oxide in the formulation. In certain embodiments, at least about 20% or at least about 30% of the polyethylene oxide in the formulation melts. Preferably, at least about 40%, or at least about 50%, or at least about 60%, or at least about 75%, or at least about 90% of the polyethylene oxide in the formulation melts during the curing step. In a preferred embodiment, about 100% of the polyethylene oxide melts.

In other embodiments, the curing step comprises subjecting the formulation to an elevated temperature for a certain period of time. In such embodiments, the curing temperature is at least as high as the softening temperature of the polyethylene oxide. According to certain embodiments, the curing temperature is at least about 60° C., at least about 62° C., ranges from about 62° C. to about 90° C., from about 62° C. to about 85° C., from about 62° C. to about 80° C., from about 65° C. to about 90° C., from about 65° C. to about 85° C. or from about 65° C. to about 80° C. The curing temperature preferably ranges from about 68° C. to about 90° C., from about 68° C. to about 85° C., from about 68° C. to about 80° C., from about 70° C. to about 90° C., from about 70° C. to about 85° C., from about 70° C. to about 80° C., from about 72° C. to about 90° C., from about 72° C. to about 85° C. or from about 72° C. to about 80° C. The curing temperature may be at least about 60° C., at least about 62° C., less than about 90° C. or less than about 80° C. Preferably, it is in the range of from about 62° C. to about 72° C. or from about 68° C. to about 72° C. Preferably, the curing temperature is at least as high as the lower limit of the softening temperature range of the polyethylene oxide, or at least about 62° C., or at least about 68° C. More preferably, the curing temperature is within the softening temperature range of the polyethylene oxide, or at least about 70° C. In further embodiments, the curing temperature is at least as high as the upper limit of the softening temperature range of the polyethylene oxide, or at least about 72° C. In further embodiments, the curing temperature is higher than the upper limit of the softening temperature range of the polyethylene oxide, or at least about 75° C., or at least about 80° C.

In those embodiments where the curing step involves subjecting the formulation to an elevated temperature for a certain period of time, this period of time is hereinafter referred to as the curing time. For the measurement of the curing time, a starting point and an end point of the curing step are defined. For the purposes of the present invention, the starting point of the curing step is defined to be the point in time when the curing temperature is reached.

In certain embodiments, the temperature profile during the curing step shows a plateau-like form between the starting point and the end point of the curing. In such embodiments, the end point of the curing step is defined to be the point in time when the heating is stopped or at least reduced, e.g. by terminating or reducing the heating and/or by starting a subsequent cooling step, and the temperature subsequently drops below the curing temperature by more than about 10° C. and/or below the lower limit of the softening temperature range of polyethylene oxide, for example, below about 62° C. When the curing temperature is reached and the curing step is thus started, deviations from the curing temperature in the course of the curing step can occur. Such deviations are tolerated as long as they do not exceed a value of about ±10° C., preferably about ±6° C., and more preferably about ±3° C. For example, if a curing temperature of at least about 75° C. is to be maintained, the measured temperature may temporarily increase to a value of about 85° C., about 81° C., or about 78° C., and the measured temperature may also temporarily drop down to a value of about 65° C., about 69° C. or about 72° C. In the cases of a larger decrease of the temperature and/or in the case that the temperature drops below the lower limit of the softening temperature range of polyethylene oxide, for example below about 62° C., the curing step is discontinued, i.e. an end point is reached. Curing can be restarted by again reaching the curing temperature.

In other embodiments, the temperature profile during the curing step shows a parabolic or triangular form between the starting point and the end point of the curing. This means that after the starting point, i.e., the point in time when the curing temperature is reached, the temperature further increases to reach a maximum, and then decreases. In such embodiments, the end point of the curing step is defined to be the point in time when the temperature drops below the curing temperature.

Depending on the apparatus used for the curing (i.e., curing device), different temperatures within the curing device can be measured to characterize the curing temperature.

In certain embodiments, the curing step may take place in an oven. In such embodiments, the temperature inside the oven is measured. Based thereon, when the curing step takes place in an oven, the curing temperature is defined to be the target inside temperature of the oven and the starting point of the curing step is defined to be the point in time when the inside temperature of the oven reaches the curing temperature. The end point of the curing step is defined to be (1) the point in time when the heating is stopped or at least reduced and the temperature inside the oven subsequently drops below the curing temperature by more than about 10° C. and/or below the lower limit of the softening temperature range of high molecular weight polyethylene oxide, for example below about 62° C., in a plateau-like temperature profile or (2) the point in time when the temperature inside the oven drops below the curing temperature in a parabolic or triangular temperature profile. Preferably, the curing step starts when the temperature inside the oven reaches a curing temperature of at least about 62° C., at least about 68° C., at least about 70° C., at least about 72° C. or at least about 75° C. In preferred embodiments, the temperature profile during the curing step shows a plateau-like form, wherein the curing temperature, i.e. the inside temperature of the oven, is at least about 68° C., about 70° C., about 72° C., about 73° C., or lies within a range of from about 70° C. to about 75° C., and the curing time is preferably in the range of from about 30 minutes to about 20 hours, from about 30 minutes to about 15 hours, from about 30 minutes to about 4 hours or from about 30 minutes to about 2 hours. In certain embodiments, the curing time is in the range of from about 30 minutes to about 90 minutes.

In certain other embodiments, the curing step takes place in curing devices that are heated by an air flow and comprise a heated air supply (inlet) and an exhaust, e.g., a coating pan or fluidized bed. Such curing devices will hereinafter be called convection curing devices. In such curing devices, it is possible to measure the temperature of the inlet air, i.e., the temperature of the heated air entering the convection curing device and/or the temperature of the exhaust air, i.e., the temperature of the air leaving the convection curing device. It is also possible to determine or at least estimate the temperature of the formulations inside the convection curing device during the curing step, e.g., by using infrared temperature measurement instruments (such as an IR gun) or by measuring the temperature using a temperature probe that was placed inside the curing device near the formulations. Based thereon, when the curing step takes place in a convection curing device, the curing temperature can be defined and the curing time can be measured as follows.

In one embodiment (method 1), the curing temperature is defined to be the target inlet air temperature and the starting point of the curing step is defined to be the point in time when the inlet air temperature reaches the curing temperature. The end point of the curing step is defined to be (1) the point in time when the heating is stopped or at least reduced and the inlet air temperature subsequently drops below the curing temperature by more than about 10° C. and/or below the lower limit of the softening temperature range of high molecular weight polyethylene oxide, for example below about 62° C., in a plateau-like temperature profile, or (2) the point in time when the inlet air temperature drops below the curing temperature in a parabolic or triangular temperature profile. Preferably, the curing step starts according to method 1, when the inlet air temperature reaches a curing temperature of at least about 62° C., at least about 68° C., at least about 70° C., at least about 72° C. or at least about 75° C. In a preferred embodiment, the temperature profile during the curing step shows a plateau-like form, wherein the curing temperature, i.e. the target inlet air temperature, is preferably at least about 72° C. For example, when the temperature is about 75° C., and the curing time which is measured according to method 1 is preferably in the range of from about 15 minutes to about 2 hours or about 30 minutes or about 1 hour.

In another embodiment (method 2), the curing temperature is defined to be the target exhaust air temperature and the starting point of the curing step is defined to be the point in time when the exhaust air temperature reaches the curing temperature. The end point of the curing step is defined to be (1) the point in time when the heating is stopped or at least reduced and the exhaust air temperature subsequently drops below the curing temperature by more than about 10° C. and/or below the lower limit of the softening temperature range of high molecular weight polyethylene oxide, (for example below about 62° C., in a plateau-like temperature profile) or (2) the point in time when the exhaust air temperature drops below the curing temperature in a parabolic or triangular temperature profile. Preferably, the curing step starts according to method 2, when the exhaust air temperature reaches a curing temperature of at least about 62° C., at least about 68° C., at least about 70° C., at least about 72° C. or at least about 75° C. In preferred embodiments, the temperature profile during the curing step shows a plateau-like form, wherein the curing temperature, i.e. the target exhaust air temperature, is preferably at least about 68° C., at least about 70° C. or at least about 72° C. For example, when the target exhaust air temperature is about 68° C., about 70° C., about 72° C., about 75° C. or about 78° C., and the curing time which is measured according to method 2 is preferably in the range of from about 1 minute to about 2 hours or from about 5 minutes to about 90 minutes. For example, the curing time is about 5 minutes, about 10 minutes, about 15 minutes, about 30 minutes, about 60 minutes, about 70 minutes, about 75 minutes or about 90 minutes. In a more preferred embodiment, the curing time which is measured according to method 2 is in the range of from about 15 minutes to about 1 hour.

In a further embodiment (method 3), the curing temperature is defined to be the target temperature of the formulations and the starting point of the curing step is defined to be the point in time when the temperature of the formulations, which can be measured for example by an IR gun, reaches the curing temperature. The end point of the curing step is defined to be (1) the point in time when the heating is stopped or at least reduced and the temperature of the formulations subsequently drops below the curing temperature by more than about 10° C. and/or below the lower limit of the softening temperature range of high molecular weight polyethylene oxide, (for example below about 62° C.), in a plateau-like temperature profile or (2) the point in time when the temperature of the formulations drops below the curing temperature in a parabolic or triangular temperature profile. Preferably, the curing step starts according to method 3, when the temperature of the formulations reaches a curing temperature of at least about 62° C., at least about 68° C., at least about 70° C., at least about 72° C. or at least about 75° C.

In still another embodiment (method 4), the curing temperature is defined to be the target temperature measured using a temperature probe such as a wire thermocouple, that is placed inside the curing device near the formulations, and the starting point of the curing step is defined to be the point in time when the temperature measured using the temperature probe reaches the curing temperature. The end point of the curing step is defined to be (1) the point in time when the heating is stopped or at least reduced and the temperature measured using the temperature probe subsequently drops below the curing temperature by more than about 10° C. and/or below the lower limit of the softening temperature range of polyethylene oxide, (for example below about 62° C.), in a plateau-like temperature profile, or (2) the point in time when the temperature measured using the temperature probe drops below the curing temperature in a parabolic or triangular temperature profile. Preferably, the curing step starts when the temperature measured using a temperature probe registers a temperature in the curing device of at least about 62° C., at least about 68° C., at least about 70° C., at least about 72° C. or at least about 75° C. In a preferred embodiment, the temperature profile during the curing step shows a plateau-like form, wherein the curing temperature is at least about 68° C. For example, when the temperature is about 70° C., and the curing time which is measured according to method 4 is preferably in the range of from about 15 minutes to about 2 hours or about 60 minutes to about 90 minutes.

If curing takes place in a convection curing device, the curing time can be measured by any of the methods described above.

In certain embodiments, the curing temperature is defined as a target temperature range. For example, the curing temperature is defined as a target inlet air temperature range or a target exhaust air temperature range. In such embodiments, the starting point of the curing step is defined to be the point in time when the lower limit of the target temperature range is reached; the end point of the curing step is defined to be the point in time when the heating is stopped or at least reduced; the temperature subsequently drops below the lower limit of the target temperature range by more than about 10° C. and/or below the lower limit of the softening temperature range of polyethylene oxide (for example, below about 62° C.).

The curing time, i.e., the time period the formulation is subjected to the curing temperature, which can, for example, be measured according to the methods described above, is at least about 1 minute or at least about 5 minutes. The curing time may vary from about 1 minute to about 24 hours, from about 5 minutes to about 20 hours, from about 10 minutes to about 15 hours, from about 15 minutes to about 10 hours or from about 30 minutes to about 5 hours depending on the specific formulation and the curing temperature. According to certain embodiments, the curing time varies from about 15 minutes to about 30 minutes. According to further embodiments, wherein the curing temperature is at least about 60° C., at least about 62° C., at least about 68° C., at least about 70° C., at least about 72° C. or at least about 75° C., or varies from about 62° C. to about 85° C. or from about 65° C. to about 85° C., then the curing time is preferably at least about 15 minutes, at least about 30 minutes, at least about 60 minutes, at least about 75 minutes, at least about 90 minutes or at least about 120 minutes. In preferred embodiments, wherein the curing temperature is, for example, at least about 62° C., at least about 68° C., at least about 70° C., at least about 72° C. or at least about 75° C., or ranges from about 62° C. to about 80° C., from about 65° C. to about 80° C., from about 68° C. to about 80° C., from about 70° C. to about 80° C. or from about 72° C. to about 80° C., then the curing time is preferably at least about 1 minute, at least about 5 minutes, at least about 10 minutes, at least about 15 minutes or at least about 30 minutes. In certain such embodiments, the curing time can be chosen to be as short as possible while still achieving the desired result (e.g., increased tamper resistance). For example, the curing time preferably does not exceed about 5 hours, does not exceed about 3 hours or does not exceed about 2 hours. Preferably, the curing time is in the range of from about 1 minute to about 5 hours, from about 5 minutes to about 3 hours, from about 15 minutes to about 2 hours, or from about 15 minutes to about 1 hour. Any combination of the curing temperatures and the curing times as disclosed herein lies within the scope of the present invention.

In certain embodiments, the composition is only subjected to the curing temperature until the polyethylene oxide present in the formulation has reached its softening temperature and/or at least partially melts. In certain such embodiments, the curing time may be less than about 5 minutes, for example, the curing time may vary from greater than 0 minutes to about 3 hours, from about 1 minute to about 2 hours or from about 2 minutes to about 1 hour. Instant curing is possible by choosing a curing device which allows for an instant heating of the polyethylene oxide in the formulation to at least its softening temperature, so that the high molecular weight polyethylene oxide at least partially melts. Such curing devices are, for example, microwave ovens, ultrasound devices, light irradiation apparatus such as UV-irradiation apparatus, ultra-high frequency (UHF) fields or any other apparatus known to the person skilled in the art.

The size of the formulation may determine the required curing time and curing temperature to achieve the desired tamper resistance.

In certain embodiments, the curing step leads to a decrease in the density of the formulation, such that the density of the cured formulation is lower than the density of the formulation prior to the curing step. Preferably, the density of the cured formulation in comparison to the density of the uncured formulation decreases by at least about 0.5%. More preferably, the density of the cured formulation in comparison to the density of the uncured formulation decreases by at least about 0.7%, at least about 0.8%, at least about 1.0%, at least about 2.0% or at least about 2.5%.

In certain embodiments, the solid, controlled-release dosage form is cured at a temperature of at least the softening point of the polyethylene oxide for at least 1 minute, at least 5 minutes or at least 15 minutes.

In other embodiments, the solid controlled-release dosage form is cured at a temperature of at least the softening point of the polyethylene oxide from about 1 minute to about 48 hours, from about 5 minutes to about 24 hours, from about 15 minutes to about 1 hour or about 30 minutes.

The solid controlled-release dosage form can be cured, e.g., at a temperature of at least about 60° C., at least about 65° C., at least about 70° C., at least about 75° C. or at a temperature of about 72° C.

In alternative embodiments, the solid controlled-release dosage form can be cured at a temperature from about 60° C. to about 90° C., from about 62° C. to about 72° C., from about 65° C. to about 85° C., from about 70° C. to about 80° C., from about 75° C. to about 80° C. or from about 70° C. to about 75° C.

### Flattening Procedures

In certain embodiments, dosage forms of the present invention may be flattened without substantially compromising the release of the opioid antagonist and agonist or the integrity of the dosage form. Flatness is described in terms of the thickness of the smallest diameter of the flattened shape compared to the thickness of the smallest diameter of the non-flattened shape. This comparison is expressed in % thickness, based on either (i) the thickness of the smallest diameter of the non-flattened shape when the initial shape is non-spherical or (ii) the thickness of the diameter when the initial shape is spherical. The thickness may be measured using a thickness gauge (e.g., a digital thickness gauge or digital caliper). The flattening force may be applied by any possible method. For purposes of testing the dosage forms of the present invention, a carver-style bench press may be used (unless otherwise specified) so as to achieve the target flatness or reduced thickness. According to certain embodiments of the invention, the flattening does not result in breaking of the dosage form into separate pieces; however, edge splits and cracks may occur.

In certain embodiments of the invention, a hammer can be used for flattening a dosage form. In such a process, hammer strikes can be manually applied from a direction substantially normal to the thickest dimension of the dosage form. The flatness is then described in the same manner as disclosed above.

In other embodiments, flattening can be measured relative to breaking strength or hardness tests, as described in Remington's Pharmaceutical Sciences, 18th edition, 1990, Chapter 89 “Oral Solid Dosage Forms”, pages 1633-1665, using the Schleuniger Apparatus. In such an embodiment, the dosage form is pressed between a pair of flat plates arranged in parallel such that the force is applied substantially normal to the thickest dimension of the dosage form, thereby flattening the dosage form. The flattening of the dosage form may be described in terms of % flattening, based on the thickness of the dimension being flattened before conducting the breaking strength test. The breaking strength (or hardness) is defined as the force at which the tested dosage form breaks. Dosage forms that do not break, but which are deformed due to a force applied are considered to be break-resistant at that particular force.

A further test to quantify the strength of dosage forms is the indentation test using a Texture Analyzer, such as the TA-XT2 Texture Analyzer (Texture Technologies Corp., 18 Fairview Road, Scarsdale, N.Y. 10583). In this method, a dosage form is placed on top of a stainless steel stand with a slightly concave surface and penetrated by the descending probe of the Texture Analyzer, such as a TA-8A ⅛ inch diameter stainless steel ball probe. Before starting the measurement, the dosage form is aligned directly under the probe, such that the descending probe will penetrate the tablet pivotally, i.e., in the center of the dosage form, and such that the force of the descending probe is applied substantially perpendicular to the diameter and substantially in line with the thickness of the dosage form. First, the probe of the Texture Analyzer starts to move towards the dosage form sample at the pre-test speed. When the probe contacts the dosage form surface and the trigger force set is reached, the probe continues its movement with the test speed and penetrates the dosage form. For each penetration depth or distance of the probe, the corresponding force is measured. When the probe has reached the desired maximum penetration depth, it changes direction and moves back at the post-test speed, while further measurements are taken. The cracking force is defined to be the force of the first local maximum that is reached in the corresponding force/distance diagram and is calculated using, for example, the Texture Analyzer software “Texture Expert Exceed, Version 2.64 English”.

The term “resistant to crushing” is defined for the purposes of certain embodiments of the present invention as referring to dosage forms that can at least be flattened with a bench press, as described above, without breaking to no more than about 60% thickness, no more than about 50% thickness, no more than about 40% thickness, no more than about 30% thickness, no more than about 20% thickness, no more than about 10% thickness or no more than about 5% thickness.

In embodiments with opioid agonists, the amount of opioid agonist released at 0.5 hour from a flattened dosage form deviates no more than about 10% points, 15% points or 20% points from the amount released at 0.5 hour from a non-flattened dosage form as measured by an in-vitro dissolution in a USP Apparatus 1 (basket) at 100 rpm in 900 ml simulated gastric fluid without enzymes (SGF) at 37° C.

In alternative embodiments, the solid controlled-release dosage form can be flattened without breaking, wherein the thickness of the dosage form after flattening corresponds to no more than about 60% of the thickness of the dosage form before flattening, no more than about 50% of the thickness of the dosage form before flattening, no more than about 40% of the thickness of the dosage form before flattening, no more than about 30% of the thickness of the dosage form before flattening, or no more than about 20% of the thickness of the dosage form before flattening.

The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### EXAMPLES

The present invention will now be more fully described with reference to the accompanying examples. It should be understood, however, that the following description is illustrative only and should not be taken in any way as a restriction of the invention.

### Example 1

### Prophetic

A 400 mg tablet (Tablet A) including 20 mg of hydrocodone bitartrate and 6.5 mg naloxone hydrochloride is prepared using high molecular weight polyethylene oxide (PEO 303-MW 7,000,000), as set forth in Table 1 below.

TABLE 1
(Tablet A)

| | Hydrocodone (mg) | Naloxone (mg) | Total wt (QS with PEO) |
|---|---|---|---|
| Core | 16 | 6 | 200 |
| Shell | 4 | 0.5 | 200 |
| Total | 20 | 6.5 | 400 |

To prepare the core, a single station Manesty Type F 3 tablet press is equipped with 7.94 mm, round, standard concave plain tooling. A powdered aliquot of the core blend, as set forth above in Table 1, is weighed out to target weight of 200 mg, charged into the die and compressed to form the core of Tablet A.

To prepare the shell, the single station Manesty Type F 3 tablet press is equipped with 10.32 mm, round, standard concave plain tooling. 100 mg of the shell blend, as set forth in Table 1, was placed in the die. The tablet core is prepared above was manually centered in the die (on top of the powder bed) and an additional 100 mg of the shell blend is placed on top of the tablet in the die. The materials are then manually compressed by turning the compression wheel to form compression coated Tablet A.

Several compression coated Tablet A tablets prepared as above are placed onto a tray, which is placed in a Hotpack model 435304 oven targeting 72° C. for 30 minutes to cure.

### Example 2

### Prophetic

A 500 mg tablet (Tablet B) including 40 mg of oxycodone hydrochloride and 4.5 naloxone hydrochloride is prepared using high molecular weight polyethylene oxide (PEO 303-MW 7,000,000), as set forth in Table 2 below.

TABLE 2
(Tablet B)

| | | Naloxone (mg) | Total wt |
|---|---|---|---|
| | Oxycodone (mg) | wt | (QS with PEO) |
| Core | 32 | 4 | 300 |
| Shell | 8 | 0.5 | 200 |
| Total | 20 | 4.5 | 500 |

To prepare the core, a single station Manesty Type F 3 tablet press is equipped with 8.73 mm, round, standard concave plain tooling. A powdered aliquot of the core blend, as set forth above in Table 2, is weighed out to target weight of 300 mg, charged into the die and compressed to form the core of Tablet B.

To prepare the shell, the single station Manesty Type F 3 tablet press is equipped with 11.11 mm, round, standard concave plain tooling. The first portion of the 200 mg shell blend, as set forth in Table 2, is placed in the die. The tablet core as prepared above was manually centered in the die (on top of the powder bed) and the remaining portion of the 200 mg shell blend is placed on top of the tablet in the die. The materials are then manually compressed by turning the compression wheel to form compression coated Tablet B.

Several compression coated Tablet B tablets prepared as above are placed onto a tray, which is placed in a Hotpack model 435304 oven targeting 72° C. for 30 minutes to cure.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

1. A solid controlled-release dosage form comprising a core comprising a core portion of an opioid antagonist and a shell encasing the core and comprising a shell portion of the opioid antagonist, wherein a release profile of the core portion of opioid antagonist is different than the release profile of the shell opioid antagonist.

2. The solid controlled-release dosage form of claim 1, wherein the release profile is defined by the amount of the opioid antagonist released from the dosage form.

3. The solid controlled-release dosage form of claim 1, wherein the release profile is defined by the release rate of the opioid antagonist released from the dosage form.

4. The solid controlled-release dosage form of claim 1, wherein the release profile is defined as the duration of release of the opioid antagonist released from the dosage form.

5. The solid controlled-release dosage form of claim 2, wherein the amount of antagonist in the core is more than the amount of antagonist in the shell.

6. The solid controlled-release dosage form of claim 2, wherein the amount of antagonist in the core is less than the amount of antagonist in the shell.

7. The solid controlled-release dosage form of claim 3, wherein the release rate of antagonist from the core is higher than the release rate of antagonist from the shell.

8. The solid controlled-release dosage form of claim 3, wherein the release rate of antagonist from the core is less than the release rate of antagonist from the shell.

9. The solid controlled-release dosage form of claim 4, wherein the duration of the release of antagonist from the core is longer than the duration of release of antagonist from the shell.

10. The solid controlled-release dosage form of claim 4, wherein the duration of the release of antagonist from the core is less than the duration of the release of antagonist from the shell.

11. The solid controlled-release dosage form of claim 1, wherein the antagonist in the core and the antagonist in the shell are independently selected from the group consisting of naltrexone, naloxone, nalmefene, cyclazacine, levallorphan, pharmaceutically acceptable salts thereof and mixtures thereof.

12. The solid controlled-release dosage form of claim 1, wherein the opioid antagonist in the core is the same as the opioid antagonist in the shell.

13. The solid controlled-release dosage form of claim 1, wherein the opioid antagonist in the core is different from the opioid antagonist in the shell.

14. The solid controlled-release dosage form of claim 1, wherein the opioid antagonist in both the core and the shell is naloxone or a pharmaceutically acceptable salt thereof.

15. The solid controlled-release dosage form of claim 1, wherein the amount of antagonist in the shell is an amount effective to deter the abuse of the dosage form.

16. The solid controlled-release dosage form of claim 1, wherein the amount of antagonist in the shell is an amount effective to deter the intravenous abuse of the dosage form.

17. The solid controlled-release dosage form of claim 1, wherein the amount of antagonist in the shell is an amount effective to deter the nasal abuse of the dosage form.

18. The solid controlled-release dosage form claim 1, further comprising an opioid analgesic.

19-72. (canceled)

73. A method of treating pain in a subject in need thereof, comprising administering to the subject a solid controlled-release dosage form claim 18.

74. A method of preparing a solid controlled-release dosage form comprising preparing a core comprising a core portion of an opioid antagonist; and encasing the core with a shell comprising a shell portion of the opioid antagonist; wherein the release profile of the core portion of opioid antagonist is different than the release profile of the shell portion of opioid antagonist.

75-76. (canceled)
